(19) United States
(12) Patent Application Publication
(10) Pub. No.: US 2019/0246881 A1
(43) Pub. Date: Aug. 15, 2019

(54) Disposable Protective Sheath for Endoscope

(71) Applicant: Jeffrey L. Aull, Jacksonville, FL (US)

(72) Inventor: Jeffrey L. Aull, Jacksonville, FL (US)

(21) Appl. No.: 16/272,720

(22) Filed: Feb. 11, 2019

Related U.S. Application Data
(60) Provisional application No. 62/629,237, filed on Feb. 12, 2018.

(51) Int. Cl. A61B 1/00; A61B 1/018

(52) U.S. Cl. CPC A61B 1/00142; A61B 1/00137; A61B 1/00103; A61B 1/018

(57) ABSTRACT

A disposable endoscope sheath having a flexible tubular member joined to a grasping member, the tubular member having a closed distal end, and an annular, elastic locking ring temporarily mounted onto the distal end of the grasping member, whereby the locking ring is rolled onto the tubular member after the sheath has been disposed onto an endoscope shaft, the size and elasticity of the locking ring being chosen such that the tubular member is securely affixed to the endoscope shaft.

### BACKGROUND OF THE INVENTION

This application relates generally to the field of protective sheaths, sleeves or similar cover devices for endoscopes, and more particularly relates to the such sheaths that are easily and securely mounted onto and removed from an endoscope, the sheaths providing protection against contamination before and during use. The sheaths are designed to be sterilizable, disposable and relatively inexpensive to manufacture.

Disposable cover sheaths for endoscopes are known, the sheaths typically comprising an elongated, thin-walled, flexible tubular member having a closed distal end and an open proximal end to receive the elongated shaft of an endoscope. The sheaths are composed of a material which may be sterilized prior to use. The sheaths are typically provided with a lens or similar transparent member at the closed end.

Most known sheaths are constructed in a manner whereby securement of the sheath onto the endoscope is accomplished through a friction, compression or mechanical fit onto the headpiece of the endoscope, the headpiece being an enlarged body from which the endoscope shaft extends. In many instances, the distal end of the headpiece presents a conical exterior configuration adjacent the proximal end of the endoscope shaft. For this headpiece design, it is known to provide a disposable sheath whose proximal end comprises a mounting member having a conical bore formed of a slightly compressible, slightly elastic material that is sized and dimensioned such that when the endoscope shaft is inserted into the sheath, the conical distal end of the headpiece mounting member is pressed into the conical bore of the sheath, whereby friction and/or compressive pressure secures the sheath onto the endoscope.

A problem with this construction is that unless the length of the sheath is accurately matched with the length of the endoscope shaft, the sheath will either be too short, such that the mounting member of the sheath cannot be positioned on the endoscope headpiece to retain the sheath on the endoscope, or the sheath will be too long, such that with the mounting member affixed onto the headpiece the distal portion of the sheath extends beyond the distal end of the endoscope shaft. In the latter case, to bring the distal closed end of the sheath into contact with the distal end of the endoscope shaft, the excessive material of the sheath would have to be gathered up to pull the distal end of the sheath into abutment with the distal end of the endoscope and held in place by hand, which is not optimal in any sense. Furthermore, in the case of a friction or compressive mating, the securing forces maintaining the sheath on the endoscope headpiece may be reduced by liquid intrusion or movement of the endoscope during the procedure.

It is an object of this invention to provide a disposable protective endoscope sheath having an improved securement mechanism that addresses the problems discussed above.

### SUMMARY OF THE INVENTION

The invention is an endoscope sheath, sleeve or similar cover member adapted to be mounted onto an endoscope whereby the shaft of the endoscope is protected from contamination during use, the sheath being constructed and designed to be disposable such that a used sheath is easily removed from an endoscope and replaced by a new sheath for a subsequent procedure. The sheath comprises in general an elongated, thin-walled, flexible tubular member having a closed distal end and an open proximal end through which the elongated shaft of an endoscope may be inserted. The open proximal end of the tubular member is joined to a grasping or manipulating member, the grasping member being open on its proximal end and having a bore sized to allow passage of the endoscope shaft therethrough. The grasping member is for manipulation and handling purposes and is not utilized to secure the sheath in place on the endoscope, and therefore may possess various internal or external configurations and may be formed of a relatively hard, non-resilient material in comparison to the material of the tubular member, which is structured to have minimal rigidity. The closed distal end of the tubular member is provided with a lens or similar transparent member so as not to interfere with the functionality of the endoscope.

The distal portion of the grasping member comprises a ring seat segment, preferably cylindrical, that is sized to temporarily retain an elastic locking ring annularly disposed thereon. The size and elasticity of the locking ring is chosen such that the locking ring is snugly retained by the ring seat segment without constricting the ring seat segment prior to securing the sheath onto the endoscope shaft yet is easily removed from the ring seat segment by rolling the locking ring in the distal direction.

To secure the sheath onto the endoscope shaft, the shaft is inserted through the grasping member and into the tubular member of the sheath such that the distal end of the endoscope shaft abuts the distal end of the sheath. Depending on the relative lengths of the sheath tubular member and the endoscope shaft, the grasping member may be located adjacent the endoscope headpiece or separated from the headpiece and situated at some point along the endoscope shaft. The locking ring is then rolled off the ring seat segment in the distal direction and onto the exterior of the distal end of the tubular member. The elasticity of the locking ring combined with the flexibility and thinness of the tubular member securely affixes the tubular member of the sheath onto the endoscope shaft in a manner that precludes axial movement during normal use of the endoscope. The length of the endoscope sheath for use on a particular endoscope is chosen to match or be less than the length of the elongated shaft of the endoscope. In this manner, the elastic locking ring secures the tubular member onto the endoscope with the proximal closed end of the sheath properly abutting the distal end of the endoscope shaft regardless of the whether the shaft covers the entire length of the endoscope shaft and without any need to mate the sheath with the endoscope headpiece. To remove the sheath after use, the locking ring is rolled in the proximal direction until it passes from the sheath tubular member and back onto the ring seat segment, such that the tubular member and grasping member are now easily slipped off the endoscope.

In alternative summary language, the invention in its embodiments is an endoscope sheath configured for use on an endoscope having an elongated shaft, said endoscope sheath comprising: a tubular member, a grasping member connected to said tubular member, and an elastic locking ring; said tubular member having an open proximal end and a closed distal end; said grasping member having a bore in communication with said open proximal end of said tubular member, said grasping member further comprising a ring seat segment adjacent said tubular member, said ring seat segment configured to receive said elastic locking ring thereon in a stretched configuration, said elastic locking ring being movable between a position on said ring seat segment to a position on said tubular member; whereby, with said endoscope sheath disposed on a shaft of an endoscope, movement of said elastic locking ring from the position on said ring seat segment to the position on said tubular member compresses said tubular member onto the endoscope shaft and secures said endoscope sheath onto said endoscope. Furthermore, such invention wherein said tubular member is an elongated, thin-walled, flexible member; wherein said tubular member is an elongated, thin-walled, elastic member; wherein said grasping member bore has a proximal end and a distal end, and said proximal end is wider than said distal end; wherein said closed distal end of said tubular member is transparent; wherein said closed distal end of said tubular member comprises a lens; wherein said grasping member has a collar member; wherein said grasping member is more rigid than said tubular member; wherein said closed distal end of said tubular member comprises an end cap of greater rigidity than said tubular member; wherein said ring seat member is cylindrical; wherein said elastic locking ring has an inner diameter and said tubular member has an outer diameter, and further wherein the inner diameter of said elastic locking ring is smaller than said outer diameter of said tubular member when said elastic locking ring is in a non-stretched configuration; and/or whereby movement of said elastic locking ring from the position on said tubular member to the position on said ring seat segment no longer secures said endoscope sheath onto said endoscope.

Alternatively, the invention in its embodiments is an endoscope sheath configured for use on an endoscope having an elongated shaft, said endoscope sheath comprising: a tubular member having an external diameter, an open proximal end and a closed distal end; a grasping member connected to said tubular member, said grasping member being more rigid than said tubular member; and an elastic locking ring having an internal diameter and further having a non-stretched configuration and a stretched configuration, wherein in said non-stretched configuration said internal diameter of said elastic locking ring is smaller than said external diameter of said tubular member; said grasping member having a bore in communication with said open proximal end of said tubular member, said grasping member further comprising a cylindrical ring seat segment adjacent said tubular member, said ring seat segment configured to receive said elastic locking ring thereon in said stretched configuration, said elastic locking ring being movable between a position on said ring seat segment and a position on said tubular member; whereby, with said endoscope sheath disposed on a shaft of an endoscope, movement of said elastic locking ring from the position on said ring seat segment to the position on said tubular member constricts said tubular member onto the endoscope shaft and secures said endoscope sheath onto said endoscope. Furthermore, such invention wherein said closed distal end of said tubular member is transparent; wherein said closed distal end of said tubular member is a lens; wherein said grasping member has a collar member; wherein said closed distal end of said tubular member comprises an end cap of greater rigidity than said tubular member; wherein said grasping member bore has a proximal end and a distal end, and said proximal end is wider than said distal end; and/or whereby movement of said elastic locking ring from the position on said tubular member to the position on said ring seat segment no longer secures said endoscope sheath onto said endoscope.

Alternatively still, the invention in its embodiments is an endoscope sheath in combination with an endoscope having an elongated shaft having an external diameter, said endoscope sheath comprising: a tubular member having an external diameter, an open proximal end and a closed distal end; a grasping member connected to said tubular member, said grasping member being more rigid than said tubular member; and an elastic locking ring having an internal diameter and further having a non-stretched configuration and a stretched configuration, wherein in said non-stretched configuration said internal diameter of said elastic locking ring is smaller than said external diameter of said tubular member and said external diameter of said elongated shaft of said endoscope; said grasping member having a bore in communication with said open proximal end of said tubular member, said bore sized to receive said elongated shaft of said endoscope therethrough, said grasping member further comprising a cylindrical ring seat segment adjacent said tubular member, said ring seat segment configured to receive said elastic locking ring thereon in said stretched configuration, said elastic locking ring being movable between a position on said ring seat segment and a position on said tubular member; whereby, with said endoscope sheath disposed on said elongated shaft of said endoscope, movement of said elastic locking ring from the position on said ring seat segment to the position on said tubular member constricts said tubular member onto said elongated shaft of said endoscope and secures said endoscope sheath onto said endoscope.

### BRIEF DESCRIPTION OF THE DRAWINGS

FIG. 1 is an illustration of a representative embodiment of the sheath mounted onto but not yet secured to the shaft of an endoscope.

FIG. 2 is an illustration of the embodiment of FIG. 1, showing the locking ring rolled onto the tubular member of the sheath to secure the sheath onto the endoscope shaft.

FIG. 3 is a cross-sectional illustration of the embodiment of the sheath of FIG. 1.

### DETAILED DESCRIPTION OF THE INVENTION

With reference to the drawings, which are provided for descriptive and illustrative purposes and are meant to be non-limiting as to the scope of the embodiments of the invention, the invention will now be described. The term “distal” as used herein shall be taken to refer to the direction toward the free ends of the endoscope shaft and sheath that are inserted into the patient's cavity during use. The term “proximal” as used herein shall be taken to refer to the opposite direction, i.e., the direction toward the portion of the endoscope and sheath that remain outside the patient's cavity during use.

In general, the invention in various embodiments is an endoscope sheath, sleeve or similar cover member 10, to be referred to herein collectively using the term “sheath”, designed, structured, constructed and adapted to be mounted onto the shaft 21 of an endoscope 20 whereby the shaft 21 of the endoscope 20 is protected from contamination during use, the sheath 10 being preferably designed to be disposable, and such that a used, non-sterile sheath 10 is easily removed from an endoscope 20 and replaced by a new sheath 10 for subsequent use of the endoscope 20. The sheath 10 comprises in general an elongated, thin-walled, flexible and/or elastic tubular member 11 having a closed distal end 31 and an open proximal end 32, the proximal end 32 and the internal diameter of the tubular member 11 being designed, sized and adapted to receive the distal end of the elongated shaft 21 of an endoscope 20 therethrough. The open proximal end 32 of the tubular member 11 is joined to a grasping member 12 which may be internally configured to enable it to extend if necessary over the distal end of the endoscope headpiece 22 in non-mating manner if necessary to properly seat the sheath 10 onto the endoscope shaft 21, although this is not preferred.

The grasping, handling or manipulating member 12, to be referred to herein collectively using the term “grasping member”, is not a fitting or connector member utilized to mechanically, frictionally or compressively mate or secure the sheath 10 in place on the endoscope 20 but is instead the means to draw the sheath 10 onto the endoscope shaft 21, and therefore may be formed with various internal or external configurations which are not a matching configuration to the distal configuration of the headpiece 22 of a given endoscope 20. Because there is no requirement for a frictional or constrictive mating with the headpiece 22, the grasping member 12 may be manufactured of a harder, non-resilient material relative to the material of composition of the tubular member 11. The closed distal end 31 of the tubular member 11 is provided with a lens or similar transparent member 18 so as not to interfere with the functionality of the endoscope 20. The closed distal end 31 of the tubular member 11 may comprise an end cap 17 formed of a material that is more rigid, thicker in wall dimension and/or more structurally sound than the material composing the tubular member 11 in order to provide increased structural integrity and prevent accidental rupture when the sheath 10 is drawn onto the endoscope shaft 21 and during use.

The grasping member 12 is most preferably formed of a plastic material that is more rigid than the material composing the tubular member 11. The grasping member 12 is provided with a bore 15 in communication with the open proximal end 32 of the tubular member 11 and being of sufficient size to allow the sheath 10 to be mounted onto the endoscope shaft 21. The bore 15 is preferably wider at its proximal end than at its distal end. The bore 25 is preferably at least partially configured in the shape of a cone or funnel to provide a wide proximal opening of greater diameter than the remainder of the bore 15 to allow for easier alignment of the endoscope shaft 21 with the bore 25. The grasping member 12 itself is not required nor intended to perform any securing function to maintain the sheath 10 on the endoscope 20. Preferably, the grasping member 12 is provided with an external collar, flange, tabs or similar axially extending member 13, to be referred to herein collectively using the term “collar”, sufficiently sized to provide a gripping or pulling surface or member for ease in mounting the sheath 10 onto the endoscope 20.

The distal portion of the grasping member 12 comprises a ring seat segment 14, preferably cylindrical in external configuration, that is adjacent the tubular member 11 and temporarily retains an elastic locking ring 16 annularly disposed thereon. The size and elasticity of the locking ring 16, preferably composed of a rubber or elastic polymer material, such silicone for example, is chosen such that the locking ring 16 is slightly stretched from its neutral configuration when situated on the ring seat segment 14 so as to be snugly retained thereon, yet is readily removable from the ring seat segment 14 by rolling the locking ring 16 in the distal axial direction onto the tubular member 11 of the sheath 10. Furthermore, the non-stretched internal diameter of the open portion of the locking ring 16 is chosen for a particular endoscope shaft 21 so as to be less than the external diameter of the tubular member 11 and of the endoscope shaft 21 disposed within the tubular member 11.

The rigidity and structural integrity of the ring seat segment 14 and the internal diameter and elastic compressive strength of the locking ring 16 are chosen such that the ring seat segment 14 and in particular the internal bore 15 is not compressed onto the endoscope shaft 21 when the locking ring 16 is seated on the ring seat segment 14, such that axial movement of the sheath 10 on the endoscope shaft 21 is not restricted or impeded. Conversely, the rigidity and structural integrity of the tubular member 11 and the elastic compressive strength of the locking ring 16 are chosen such that the locking ring 16 compresses and constricts the tubular member 11 tightly onto the endoscope shaft 21, thereby retaining the sheath 10 in proper position on the endoscope shaft and preventing undesirable axial movement of the sheath 10 relative to the endoscope shaft 21.

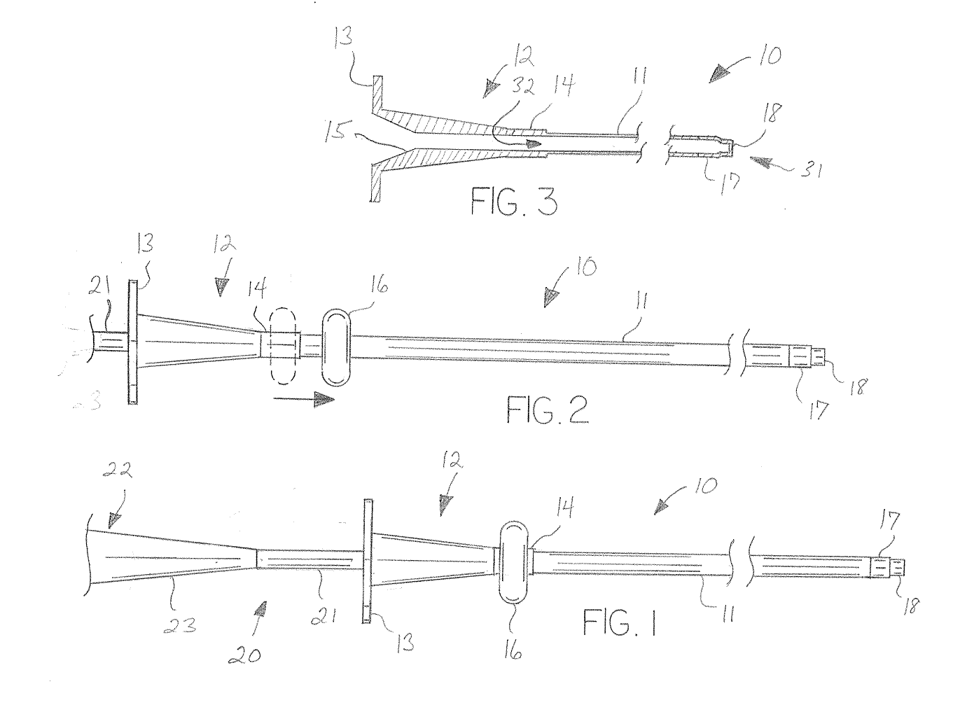

To secure the sheath 10 onto the endoscope shaft 21, the free distal end of the shaft 21 is inserted through the bore 15 of the grasping member 12 and into the tubular member 11 of the sheath 10 (or alternatively speaking, the grasping member 12 and tubular member 11 of the sheath 10 are pulled onto the endoscope shaft 21 toward the headpiece 22), as shown in FIG. 1, to the point where the distal end of the endoscope shaft 21 abuts the closed distal end 31 of the tubular member 11. The locking ring 16 is then rolled off the ring seat segment 14 onto the exterior of the distal end of the tubular member 11, as shown in FIG. 2. The size and elasticity of the locking ring 16 compressively secures the tubular member 11 of the sheath 10 onto the endoscope shaft 21 to preclude movement of the tubular member 11 in the axial direction relative to the endoscope shaft 21. The length of the endoscope sheath 10 for use on a particular endoscope 20 is chosen to be equal or lesser than the length of the elongated shaft 21 of the endoscope 20. Since the grasping member 12 does not need to abut, connect to or mount onto the endoscope headpiece 22 to secure the sheath 10 in place, a single sheath 10 will fit multiple lengths of endoscopes shafts 21 because the elastic locking ring 14 secures the tubular member 11 onto the endoscope shaft 21 with the closed distal end 31 of the sheath 10 properly abutting the distal end of the endoscope shaft 21. Since the grasping member 12 does not need to be adjacent the endoscope headpiece 22, the locking ring 16 is functional when positioned at any point along the endoscope shaft 21. To remove the sheath 10 after use, the locking ring 16 is rolled in the proximal direction until it passes off of the tubular member 111 and onto the ring seat segment 14. With the constriction and compression now removed from the tubular member 11, the used sheath 10 is now easily slipped from the endoscope 20 for replacement by a new sheath 10.

It is contemplated and understood that equivalents and substitutions for certain elements set forth above may be obvious to those of ordinary skill in the art, and therefore the true scope and definition of the invention in its various embodiments is to be as set forth in the following claims.

I claim:

1. An endoscope sheath configured for use on an endoscope having an elongated shaft, said endoscope sheath comprising:
a tubular member, a grasping member connected to said tubular member, and an elastic locking ring;
said tubular member having an open proximal end and a closed distal end;
said grasping member having a bore in communication with said open proximal end of said tubular member, said grasping member further comprising a ring seat segment adjacent said tubular member, said ring seat segment configured to receive said elastic locking ring thereon in a stretched configuration, said elastic locking ring being movable between a position on said ring seat segment to a position on said tubular member;
whereby, with said endoscope sheath disposed on a shaft of an endoscope, movement of said elastic locking ring from the position on said ring seat segment to the position on said tubular member compresses said tubular member onto the endoscope shaft and secures said endoscope sheath onto said endoscope.

2. The endoscope sheath of claim 1, wherein said tubular member is an elongated, thin-walled, flexible member.

3. The endoscope sheath of claim 1, wherein said tubular member is an elongated, thin-walled, elastic member.

4. The endoscope sheath of claim 1, wherein said grasping member bore has a proximal end and a distal end, and said proximal end is wider than said distal end.

5. The endoscope sheath of claim 1, wherein said closed distal end of said tubular member is transparent.

6. The endoscope sheath of claim 1, wherein said closed distal end of said tubular member comprises a lens.

7. The endoscope sheath of claim 1, wherein said grasping member has a collar member.

8. The endoscope sheath of claim 1, wherein said grasping member is more rigid than said tubular member.

9. The endoscope sheath of claim 1, wherein said closed distal end of said tubular member comprises an end cap of greater rigidity than said tubular member.

10. The endoscope sheath of claim 1, wherein said ring seat member is cylindrical.

11. The endoscope of claim 1, wherein said elastic locking ring has an inner diameter and said tubular member has an outer diameter, and further wherein the inner diameter of said elastic locking ring is smaller than said outer diameter of said tubular member when said elastic locking ring is in a non-stretched configuration.

12. The endoscope sheath of claim 1, whereby movement of said elastic locking ring from the position on said tubular member to the position on said ring seat segment no longer secures said endoscope sheath onto said endoscope.

13. An endoscope sheath configured for use on an endoscope having an elongated shaft, said endoscope sheath comprising:
a tubular member having an external diameter, an open proximal end and a closed distal end;
a grasping member connected to said tubular member, said grasping member being more rigid than said tubular member;
and an elastic locking ring having an internal diameter and further having a non-stretched configuration and a stretched configuration, wherein in said non-stretched configuration said internal diameter of said elastic locking ring is smaller than said external diameter of said tubular member;
said grasping member having a bore in communication with said open proximal end of said tubular member, said grasping member further comprising a cylindrical ring seat segment adjacent said tubular member, said ring seat segment configured to receive said elastic locking ring thereon in said stretched configuration, said elastic locking ring being movable between a position on said ring seat segment and a position on said tubular member;
whereby, with said endoscope sheath disposed on a shaft of an endoscope, movement of said elastic locking ring from the position on said ring seat segment to the position on said tubular member constricts said tubular member onto the endoscope shaft and secures said endoscope sheath onto said endoscope.

14. The endoscope sheath of claim 13, wherein said closed distal end of said tubular member is transparent.

15. The endoscope sheath of claim 13, wherein said closed distal end of said tubular member is a lens.

16. The endoscope sheath of claim 13, wherein said grasping member has a collar member.

17. The endoscope sheath of claim 13, wherein said closed distal end of said tubular member comprises an end cap of greater rigidity than said tubular member.

18. The endoscope sheath of claim 13, wherein said grasping member bore has a proximal end and a distal end, and said proximal end is wider than said distal end.

19. The endoscope sheath of claim 13, whereby movement of said elastic locking ring from the position on said tubular member to the position on said ring seat segment no longer secures said endoscope sheath onto said endoscope.

20. An endoscope sheath in combination with an endoscope having an elongated shaft having an external diameter, said endoscope sheath comprising:
a tubular member having an external diameter, an open proximal end and a closed distal end;
a grasping member connected to said tubular member, said grasping member being more rigid than said tubular member;
and an elastic locking ring having an internal diameter and further having a non-stretched configuration and a stretched configuration, wherein in said non-stretched configuration said internal diameter of said elastic locking ring is smaller than said external diameter of said tubular member and said external diameter of said elongated shaft of said endoscope;
said grasping member having a bore in communication with said open proximal end of said tubular member, said bore sized to receive said elongated shaft of said endoscope therethrough, said grasping member further comprising a cylindrical ring seat segment adjacent said tubular member, said ring seat segment configured to receive said elastic locking ring thereon in said stretched configuration, said elastic locking ring being movable between a position on said ring seat segment and a position on said tubular member;
whereby, with said endoscope sheath disposed on said elongated shaft of said endoscope, movement of said elastic locking ring from the position on said ring seat segment to the position on said tubular member constricts said tubular member onto said elongated shaft of said endoscope and secures said endoscope sheath onto said endoscope.
